(19) United States
(12) Patent Application Publication
(10) Pub. No.: US 2006/0105397 A1
(43) Pub. Date: May 18, 2006

(54) Method for the detection of stress biomarkers including cortisol by fluorescence polarization

(76) Inventors: Malford E. Cullum, Grayslake, IL (US); Christopher A. Duplessis, Chesapeake, CT (US); Loring J. Crepeau, Groton, CT (US)

Correspondence Address: NAVAL MEDICAL RESEARCH CENTER;ATTN: (CODE 00L), 503 ROBERT GRANT AVENUE, SILVER SPRING, MD, 20910-7500, US

(21) Appl. No.: 11/328,486

(22) Filed: Jan. 4, 2006

Related U.S. Application Data
(63) Continuation-in-part of application No. 10/700,868, filed on Nov. 5, 2003.

(51) Int. Cl. G01N 33/53

(52) U.S. Cl. 435/7.1

(57) ABSTRACT

The inventive subject matter relates to a competitive method measuring stress biomarkers in bodily fluids including serum, urine and oral fluids including saliva. The inventive method measures biomarkers including cortisol, melatonin and secretory IgA by fluorescence polarization fluorescence lifetime analysis or fluorescence resonance energy transfer.

### CROSS REFERENCE TO RELATED APPLICATION

This application is a continuation in part and claims priority to U.S. non-provisional application Ser. No. 10/700,868 filed Nov. 5, 2003. The contents of nonprovisional application Ser. No. 10/700,868 is incorporated herein by reference.

### BACKGROUND OF INVENTION

1. Field of the Invention

The inventive subject matter relates to a competitive a fluorescence method for estimating the concentration of stress biomarkers such as cortisol, melatonin and secretory IgA, in bodily fluids including serum, urine and oral fluids including saliva. The method contemplates the use of fluorescence polarization (FP), fluorescence lifetime (FLT) analysis or fluorescence resonance energy transfer (FRET).

2. Description of the Related Art

Oral fluids have been increasingly recognized as acceptable alternatives to serum for use in diagnostic tests for certain hormones, drugs, antibodies and antigens (Kraus and Konno, 1965; Hirschman and Kresge, 2001; Hofman, 2001; Tenovuo, 1989). Salivary assays will likely be significantly utilized in the detection and diagnosis of periodontal disease (Kaufman and Lamster, 2000) as well as other important infectious and noninfectious diseases (Streckfus and Bigler, 2002; Rossomando, et al, 2001; Tabak, 2001).

Saliva (Kagami, et al, 2000) and oral fluid (Cordeiro, et al, 1993) are biochemically distinct from other body fluid sources distinct. They generally reflect the serum pool, but neither saliva nor oral fluid is a passive ultrafiltrate in that no ATP utilization is required (Rehak, et al, 2000). The presence of mucins, the polyanionic glycoproteins that increase salivary viscosity, and oral flora have been largely responsible for the lack of popularity of oral fluids in clinical research. Saliva presents several challenges for diagnostics: limited reference values have been published and the standardization of sample collection has fallen behind serum (Soderling, 1989). Standardized saliva (Seymour, et al 1995) and oral fluid (Goldstein, et al, 1994) collection devices have only recently become available and should contribute to further investigations.

Antibody containing oral fluids, gingival crevicular fluid (GCF) and oral mucosal transudate (OMT) arise due to hydrostatic pressure of the capillaries and venules associated with the lingual or buccal epithelium. They offer less variation than saliva and the best alternative to serum for antibody detection (McKie, et al, 2002). GCF is similar to serum in protein composition but is significantly lower in protein concentration, being about 3% of the protein levels in blood (Marcus, et al, 1985; Burke, et al, 2002). GCF volume is about one percent of total saliva volume in the healthy mouth (Slots and Taubman, 1991). GCF is obtained by inserting an absorbent paper into the pocket or sulcus of a tooth (between the tooth and gingiva) after clearing the supragingival plaque (Thieme, et al, 1998). Its medical and dental use has not been reviewed since the 1970's (21, 22). OMT is 3-4 fold higher in protein concentration than saliva based on the IgG obtained using this device and is collected by placing a thick pad against the buccal mucosal surface juxtaposed between the parotid duct and the gingival crest (Cordeiro, et al, 1993). A current OMT device uses a salt-impregnated pad that is subsequently treated to release the antibody-containing fluid and retain the glycoproteins on the pad yielding “oral fluid”.

The oral cavity as an immunological entity has been reviewed with respect to oral diseases (Roitt and Lehner, 1981) and microbiology (Slots and Taubman, 1991). The use of oral fluids in diagnostic immunology includes detection of infection by HIV (Gallo, et al, 1997); measles, mumps and rubella (Thieme, et al, 1994); hepatitis A (Bull, et al, 1989); B (Parry, et al, 1989); and C (Sherman, et al, 1994); Helicobacter pylori (Patel, et al, 1994); dengue (Parry, et al, 1987); and Chagas' disease (Barros, et al, 1999). The current review will focus on the use of fluorescence polarization (FP)-based tests to detect antibodies to anthrax vaccine and to tuberculosis exposure using saliva, GCF or OMT as a test fluid.

In addition to infectious disease detection, identification of stress, especially in high stress occupations such as the military, is an important health issue. Detection biomarkers predictive of fatigue, stress and alertness levels would markedly enhance job performance. A number of studies have established a relationship between stress exposure, (social crowding, shift work-circadian desynchronization, altered sleep schedules, and confinement) and salivary biomarkers for cortisol, melatonin and secretory IgA (sIgA). The result is concomitant risk to health and performance. Chronic stress appears to contribute to immunologic dysfunction, malignancies, upper respiratory tract infections, gastrointestinal illness, anxiety, depression, diminished memory, decreased psychomotor function, alertness, vigilance, concentration, learning ability, cognitive performance, reflexive action and reaction time (Chouker, et al, 2002; Cohen, et al, 1991; Chrousos, 2000; Mohren, et al, 2002; Fu and Lee, 2003; Stevens and Rea, 2001; Knuttson, 2003; Schernhammer, et al, 2003; Kirschbaum and Hellhammer, 1994).

Real-time biomarker assays would be highly beneficial in detection of patient responses to stress and for the development of models that predict stress-mediated immunosuppression, creating windows of opportunity for prevention and treatment. In addition, these models could be employed to identify optimal watch-standing schedules and stress-mitigating procedures that foster improved circadian entrainment and reduced stress levels. This strategy would result in reduced fatigue, higher alertness, increased operational performance, and situational awareness.

Currently excepted methods for the clinical evaluation of markers in serum or oral fluids from patients include enzyme-linked immunosorbent assays (ELISA), agglutination or radioimmunoassays (RIA). However, these methods suffer from a number of disadvantages including interference from other molecules in the fluid environment. Because of the importance of biomarkers in stress analysis and because of the inherent deficiencies of other assay methods, a competitive fluorescence polarization (FP) assay method was developed for the detection of cortisol in bodily fluids, including saliva. FP has distinct advantages over previous methods.

Fluorescent polarization (FP) technology permits rapid, real-time, sensitive evaluation of fluid phase antigens with high specificity (Kowski and Wu, 2000). FP is predicated on the principle that fluorescent molecules emit polarized fluorescence when they absorb polarized light at a specific wavelength. However, inherent in molecules in solution is their tendency to rotate. When polarized light strikes the molecules in solution, the emitted light does not remain polarized because the molecule is rotating rapidly in solution. Therefore, in FP based assays polarized incident visible or ultraviolet light that illuminates a fluorochrome causes subsequent polarized fluorescence with emission at a longer wavelength. However, molecules in solution are capable of rotation. Polarized light striking a fluorescent molecule loses polarization due to rotation of the molecule with the rate of rotation dependent on the size of the molecule. Therefore, solutions containing slower turning, large molecule-fluorochrome complexes tends to stay polarized longer compared to smaller labeled molecules. Therefore, an antigen/antibody complex will have an inherently slower rate of rotation causing more of the polarized fluorescence to be emitted in the same plane as the incident light. In order to accommodate molecules of different sizes (up to 107 kDa molecular weight), different fluorochromes can be selected (Terpetschnig, et al, 1995).

Combining fluorochrome-labeled antigen or peptide with antibody results in an increase in FP, as measured in arbitrary millipolarization (mP) units. The smaller the fluorescent antigen, the greater the increase in mP units that is measured upon binding to its corresponding antibody, since mP depends upon the partial specific volume (approximate molecular weight in solution) of the labeled substance. The dependence is non-linear but is describable in a Perrin equation.

FP antigen-antibody binding assays require only the mixing of fluorescent reagent (antigen) with the sample (containing antibody) in a liquid buffer. In a rapid diagnostic format, essentially two FP readings are necessary; a base-line reading and a reading after a specified time. The FP value increases as binding of antigen and antibody occurs in a direct binding assay. The difference in FP between a fluorescent antigen of 10 kDa initially and the fluorescent complex consisting of it and IgG, for example, results in a measurable association using less than saturating antibody concentrations (Tencza, et al, 2000).

Because FP is conducted in a fluid environment and because polarization is a general property of fluorescent molecules, FP assays have the potential to be less susceptible to non-specific interactions occurring at the cell surface and to interferences present in non-homogeneous sample fluids. Furthermore, salivary and oral fluid assays, as opposed to the use of serum, have been increasingly recognized as a better, non-invasive alternative to serum-based diagnostics in detecting certain hormone, drug, antibody and antigen detection (Rossomando, et al, 2001; Tabak, 2001).

Other advantages of FP technology is that FP assays are ratio-metric and are thus independent of concentration. This trait allows for a theoretical requirement of two molecules to assay and is the basis for requiring no wash steps in the procedure. FP can accommodate relatively cloudy solutions, such as oral fluids or serum, without the need for time-intensive or expensive purification or clarification schemes. Furthermore, FP assays can be designed to accommodate significant variation in pH in fluid samples, such as in some media or in saliva or urine, by utilizing different pH-independent fluorochromes (U.S. Pat. No. 5,804,395 to Schade and Jolley).

In light of the advantageous properties afforded by FP assay technology, a competitive method for the detection of cortisol was developed. The assay is suitable for the detection of cortisol in oral fluids, including saliva as well as other bodily fluids such as serum.

### SUMMARY OF THE INVENTION

An object of the invention is an assay method for the detection and quantitation of cortisol and other biomarkers of stress such as melatonin and secretory IgA (sIgA) by fluorescent polarization (FP), fluorescence lifetime (FLT) analysis or fluorescence resonance energy transfer (FRET) in serum or oral fluids, including saliva.

A still further object of the invention is a rapid, real-time detection method for monitoring cortisol in individuals potentially suffering from stress.

An additional object of the invention is an assay method for the detection and quantitation of cortisol by competitive fluorescence polarization, fluorescence lifetime (FLT) analysis or fluorescence resonance energy transfer (FRET) in bodily fluids such as oral fluids, including saliva, urine and serum.

### BRIEF DESCRIPTION OF THE DRAWINGS

FIG. 1. Histogram of fluorescence polarization values in an unstimulated population.

FIG. 2. Titration of fluorescein-labeled cortisol with graded amounts of cortisol-specific antibody.

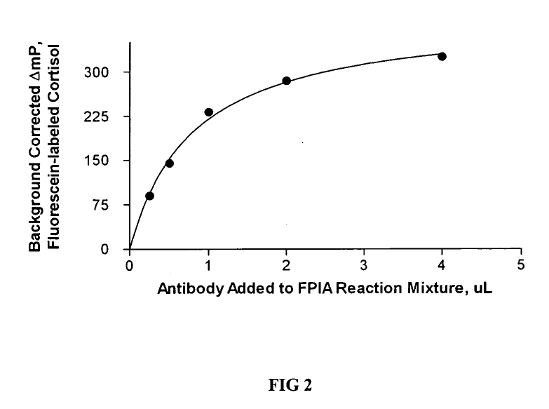

FIG. 3. Back-titration of fluorescein-labeled cortisol with unlabeled cortisol standards.

FIG. 4. Comparison of FP verses ELISA.

### DETAILED DESCRIPTION OF PREFERRED EMBODIMENTS

A number of biomarkers for stress have been identified, including cortisol, melatonin and secretory IgA (sIgA). The current application utilizes FP, or other fluorescence technology, such as FLT, and FRET, technology in a competitive assay to detect and quantitate stress-related markers. The general scheme of the assay includes the following steps:
a. intermixing a fluid sample such as oral fluid, urine or serum and a with a biomarker-specific monoclonal or polyclonal antibody;
b. obtaining a background FP measurement to blank endogenous polarized fluorescence;
c. adding set amount of fluorochrome-labeled biomarker competitor;
d. incubating the fluorochrome-labeled biomarker competitive reagent, biological sample and specific antibody for 15 seconds to 5 minutes, depending on suspected concentration of the target antigen in the sample;
e. detecting the binding interaction of the biomarker and specific antibody.
f. quantitating the concentration of the biomarker as a result of the interaction of the biomarker, biomarker competitor and an antibody that interacts specifically with all three.

Detection of specific agent would then be by either a change in fluorescence polarization or a change in fluorescence lifetime, depending on whether the technology incorporated was FP or FLT, respectively. If the method incorporated FRET, then detection is by sensitized fluorescence of the acceptor or by quenching of donor fluorescence or by fluorescence depolarization (Cui, et al, 2003; Morrison, 1988).

As a specific example the detection of cortisol in either serum or oral or salivary fluids is disclosed. Unlike total serum cortisol, which is composed of a combination of “non-active” carrier-bound and free cortisol, salivary cortisol comprises “biologically active” free cortisol. Because salivary cortisol is equilibrated into the salivary extravascular pool, its concentration is independent of saliva flow and is proportional to circulating levels making it an ideal candidate for non-invasive sampling (Kirschbaum and Hellhammer, 1994). Urinary free cortisol is a more accurate reflection of cortisol secretion than a single serum specimen (Watts and Keffer, 1989). Urinary cortisol reflects the portion of serum-free cortisol filtered by the kidney and correlates with cortisol secretion rate (Tsigos and Chrousos, 1996). The procedure for measuring cortisol by FP is essentially as described above but using fluorochrome-labeled cortisol as the competitive reagent and either polyclonal or monoclonal antibody specific to cortisol.

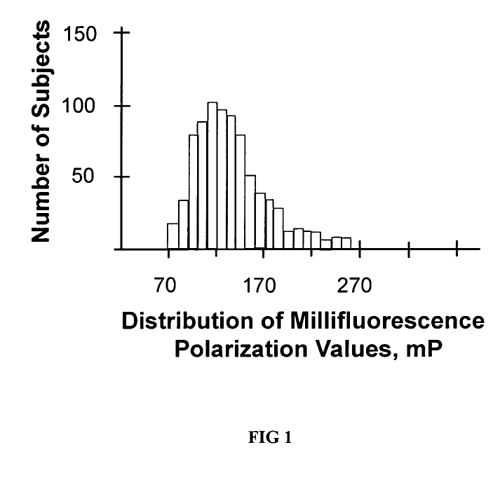

FIG. 1 shows a histogram of fluorescence polarization values of a morning, unstimulated small population (n=902). The results in FIG. 1 show a skewed distribution with a molecular mass range from approximately 800 Da to 10,000 Da and an average hydrated size of approximately 1,200 Da. (mean, 136 mP; standard deviation, 40.4 mP; median, 129 mP) assuming spherical shape, a viscosity equal to that of water (=1) at 20 degrees centigrade and one atmosphere of pressure (standard temperature and pressure). This variance among samples dictates that each individual sample must be used to background subtract intrinsic polarized fluorescence that arises from each subject's saliva sample.

An aspect of the invention is that different fluorochromes can be utilized in order to optimize results, including the incorporation of pH-independent fluorochromes. The fluorochromes that are included as an aspect of the invention include: 7-AAD, Acridine Orange, Alexa 488, Alexa 532, Alexa 546, Alexa 568, Alexa 594, Aminonapthalene, Benzoxadiazole, BODIPY 493/504, BODIPY 505/515, BODIPY 576/589, BODIPY FL, BODIPY TMR, BODIPY TR, Carboxytetramethylrhodamine, Cascade Blue, a Coumarin, Cy2, CY3, CY5, CY9, Dansyl Chloride, DAPI, Eosin, Erythrosin, Ethidium Homodimer II, Ethidium Bromide, Fluorescamine, Fluorescein, FTC, GFP (yellow shifted mutants T203Y, T203F, S65G/S72A), Hoechst 33242, Hoechst 33258, IAEDANS, an Indopyras Dye, a Lanthanide Chelate, a Lanthanide Cryptate, Lissamine Rhodamine, Lucifer Yellow, Maleimide, MANT, MQAE, NBD, Oregon Green 488, Oregon Green 514, Oregon Green 500, Phycoerythrin, a Porphyrin, Propidium Iodide, Pyrene, Pyrene Butyrate, Pyrene Maleimide, Pyridyloxazole, Rhodamine 123, Rhodamine 6G, Rhodamine Green, SPQ, Texas Red, TMRM, TOTO-1, TRITC, YOYO-1, vitamin B12, flavin-adenine dinucleotide, and nicotinamide-adenine dinucleotide.

To compare FP and ELISA measurement of cortisol, we monitored 40 subjects' sleep, using Actigraph Sleep Watches and a sleep-scoring algorithm (Cole, et al, 1992) that quantifies sleep each time the submariner gets in his bunk. Before and after each watch, we determined subjective sleepiness and mood sleepiness (Hoddes, et al, 1973) and mood (McNair, et al, 1992) using standardized questionnaires; and cognitive performance using a computer-driven assessment battery. We also collected five body temperature readings interspersed throughout the waking hours (upon awakening, prior to sleep, before and after watch sections, and a mid watch level). Finally, we collected salivary samples at each meal period from ten of the 40 subjects for subsequent cortisol analysis, to assess the circadian cycles and stress-induced and stress physiology sustained under the respective schedules.

Data were collected for 33 days. Saliva samples were collected at 0530, 1130, 1730, and 2330, using Sali-Saver® Saliva collection tubes and device (ALPCO, Windham, N.H.). Saliva samples were immediately stored in the submarine freezer with a temperature of −20° C., until it was shipped on dry ice to the NIDBR facility for −80° C. storage until analysis.

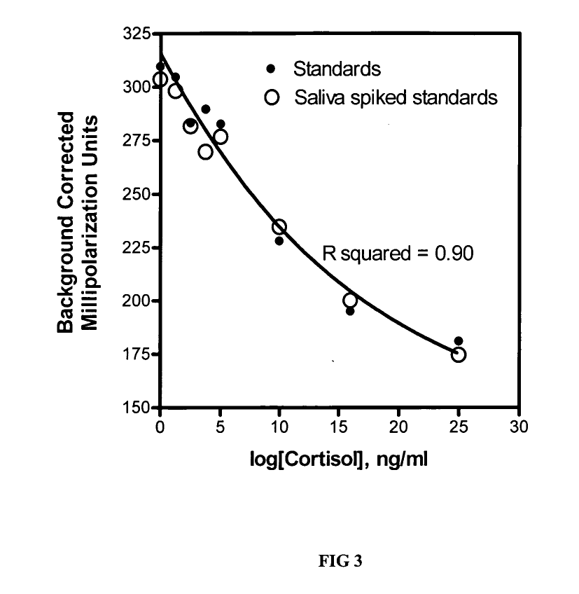

Cortisol measurement by ELISA was conducted by preparing aliquots of 100 μl for replicate analysis utilizing the Salivary Cortisol Kit. ELISA (ALPCO Diagnostics). Results were validated using independent three range calibrators (BioRad LymphoChek® 371, 372, 373, Richmond, Calif.). The ELISA kit was supplied with six standards in the ng/ml range: 0, 2, 5, 10, 20, 40 and 80 ng/ml (0-8 ug/dL, 0-220 nM). Concomitantly, FP analysis was undertaken by titration of 1.25% solution of fluorescein-labeled cortisol (Abbott AxSym® Cortisol Reagent Pack item T or any other cortisol preparation labeled in the 3 position may be used) (Pourfaraneh, et al, 1980*)) in Abbott FPIA dilution buffer with a mixture of polyclonal and monoclonal antibody (Abbott Reagent Pack item S, or any other monoclonal antibody specific to cortisol and having minimal cross-reactivity to 11-deoxycortisol, prednisolone, corticosterone 11-deoxycorticosterone, progesterone, 17-hydroxyprogesterone, testosterone, estradiol, estriol and danazol, as defined by 50% displacement of cortisol-3-I¹²⁵. The results of this analysis is illustrated in FIG. 3. The X-axis denotes the amount of antibody (item S) used with respect to the standard amount added by the Abbott AxSym® or TDx® instrument. The Y-axis denotes the background corrected millipolarization scale (0-300 mP).

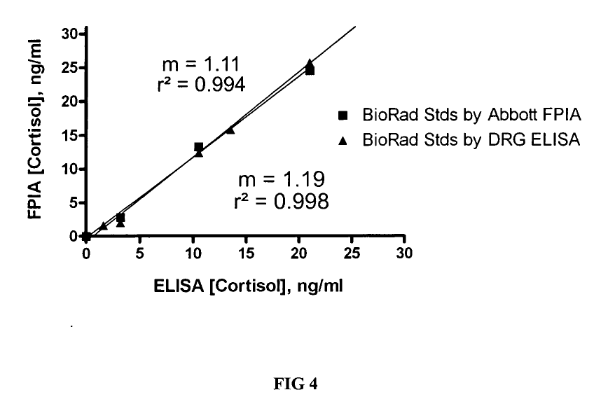

In order to further validate the standardization between FPIA and ELISA, Lymphochek® Immunoassay Plus® Control Levels 1, 2 and 3 (Bio-Rad Laboratories, Irvine, Calif.) standards were tested via FPIA and ELISA. The validation of salivary cortisol FPIA to the currently accepted gold standard salivary cortisol ELISA is accomplished by use of cross validated standards such as BioRad standards spiked into the saliva matrix, BioRad standards performed in both assay systems, and ELISA and FPIA standards spiked into a cortisol-quantitated saliva matrix. The results of this study are illustrated in FIG. 4. As shown in FIG. 4, both methods report the standards to be equivalent. The slope of the regression lines is equivalent (m=1.11-1.19 with r²=0.994-0.998) indicating that universal standards in both systems behave similarly.

### REFERENCES

1. Barros, M., A. N. Duarte Neto, V. R. A. Pereira, M. Nakazawa, W. V. Souza, Y. M. Gomes and R. Martinez. 1999. Evaluation of oral mucosal transudate for immunodiagnosis of Chagas' disease, Rev Inst Med Trop Sao Paulo 41:265-6.
2. Bull, A. R., K. J. Kimmance, J. V. Parry, K. R. Perry. 1989. Investigation of an outbreak of hepatitis A simplified by salivary antibody testing, Epidemiol Infect 103:371-6.
3. Burke, J. C., C. A. Evans, T. R. Crosby, M. I. Mednieks. 2002. Expression of secretory proteins in oral fluid after orthodontic tooth movement, Am J Orthod Dentofacial Orthop 121:310-5.
4. Cimasoni, G. 1974. The crevicular fluid, Monographs in oral science, Vol. 3, 122, S. Karger, Basel, New York.
5. Cohen, S., D. A. Tyrrell, and A. P. Smith. 1991. Psychological Stress and Susceptibility to the Common Cold. N Engl J Med, 1991; 325:606-612.
6. Chouker, A., L. Smith, F. Christ, I, Larina, I, Nichiporuk, V. Baronov, E. Bobrovnik, L. Patushkova, K. Messmer, K. Peter, and M. Thiel. 2002. Effects of Confinement (110 and 240 Days) on Neuroendocrine Stress Response and Changes of Immune Cells in Men. J Appl Physiol, 92:1619-1627.
7. Chrousos, G. P. 2000. Stress, Chronic Inflammation, and Emotional and Physical Well-Being: Concurrent Effects and Chronic Sequelae. J Allergy Clin Immunol, 106:S275-291.
8. Cole, R. J., D. F. Kripke, W. Gruen, D. J. Mullaney, and C. J. Gillin. 1992. Automatic sleep/wake identification from wrist activity. Sleep, 15:461-436.
9. Cordeiro, M. L., C. S. Turpin, S. A. McAdams. 1993. A comparative study of saliva and OraSure oral fluid, Ann N Y Acad Sci 694:330-1.
10. Cui, H. H., J. G. Valdez, J. A. Steinkamp, H. A. Crissman, H. A. 2003. Fluorescence lifetime-based discrimination and quantification of cellular DNA and RNA with phase-sensitive flow cytometry. Cytometry 52A(1): 46-55.
11. Fu, L. and C. C. Lee. 2003. The Circadian Clock: Pacemaker and Tumor Suppressor. Nature Reviews Cancer, 3:350-361.
12. Gallo, D., J. R. George, J. H. Fitchen, A. S. Goldstein, M. S. Hindahl. 1997. Evaluation of a system using oral mucosal transudate for HIV-1 antibody screening and confirmatory testing. OraSure HIV Clinical Trials Group, JAMA 277:254-8.
13. Goldstein, A. S., S. Gadojdea, D. F. Zogg. 1994. Oral collection device and method for immunoassay: U.S. Pat. No. 5,335,673. Patent and Trademark Office, United States, Epitope, Inc., Beaverton, Oreg.
14. Hirschman JD, J. A. Kresge. 2001. Synthesis of a symposium: innovative non- or minimally-invasive technologies for monitoring health and nutritional status in mothers and young children, J Nutr 131:1643S-5S.
15. Hoddes, E., V. Zarcone, H. Smythe, R. Phillips and W. C. Dement. 1973. Quantification of sleepiness: A new approach. Psychophysiology, 10:431-436.
16. Hofman L. F. 2001. Human saliva as a diagnostic specimen, J Nutr 2001 131:1621 S-5S.
17. Kaufman E., I. B. Lamster. 2000. Analysis of saliva for periodontal diagnosis—a review, J Clin Periodontol 27:453-65.
18. Kagami, H., Y. Hiramatsu, S. Hishida, Y. Okazaki, K. Horie, Y. Oda, and M. Ueda. 2000. Salivary growth factors in health and disease, Adv Dent Res 14:5-102.
19. Kirschbaum, C. and D. H. Hellhammer. 1994. Salivary cortisol in psychoneuroendocrine research: Recent developments and applications. Psychoneuroendocrinology, 19:313-333.
20. Kowski, T., J. Wu. 2000. Fluorescence polarization is a useful technology for reagent reduction in assay miniaturization, Comb Chem High Throughput Screen, 3:437-44.
21. Knuttson, A. 2003. Health Disorders of Shiftworkers. Occup Med, 53:103-108.
22. Kraus F., J. Konno. 1965. The salivary secretion of antibody, Alabama J Med Sci 2:15-22.
23. Lehner, T. 1977. The Borderland between caries and periodontal disease: Proceedings of a conference of Royal Society of Medicine, D631, Academic Press, London, New York.
24. Marcus, E. R., C. P. Jooste, H. S. Driver, J. Hattingh. 1985. The quantification of individual proteins in crevicular gingival fluid, J Periodontal Res 20:444-9.
25. McKie, A., A. Vyse, C. Maple. 2002. Novel methods for the detection of microbial antibodies in oral fluid, Lancet Infect Dis 2:18-24.
26. McNair, D. M., M. Lorr and L. F. Droppleman. 1992. Manual: Profile of mood states-revised. Education and industrial testing service, 27 pp.
27. Mohren, D. C., N. W. Nansen, I. J. Kant, J. Galama, P. A. Van Den Brandt, and G. M. Swaen. 2002. Prevalence of Common Infections among Employees in Different Work Schedules. J Occup Environ Med, 44:1003-1011.
28. Morrison, L. E. 1988. Time-resolved detection of energy transfer: theory and application to immunoassays. 174(1): 101-20.
29. Parry, J. V., K. R. Perry, S. Panday, P. P. Mortimer. 1989. Diagnosis of hepatitis A and B by testing saliva, Journal Med Virol 28:255-60.
30. Parry, J. V., K. R. Perry, P. P. Mortimer. 1987. Sensitive assays for viral antibodies in saliva: an alternative to tests on serum, Lancet 2:72-5.
31. Patel, P., M. Mendall, S. Khulusi S, N. Molineaux, J. Levy, J. D. Maxwell and T. C. Northfield. 1994. Salivary antibodies to Helicobacter pylori: screening dyspeptic patients before endoscopy, Lancet 344:511-4.
32. Pourfarzaneh, M., G. W. White, J. Landon, D. S. Smith. 1980. Cortisol directly determined in serum by fluoroimmunoassay with magnetizable solid phase, Clin Chem. 26:730-733.
33. Rehak, N. N., S. A. Cecco, G. Csako. 2000. Biochemical composition and electrolyte balance of “unstimulated” whole human saliva, Clin Chem Lab Med 38:335-43.
34. Roitt, I. M., T. Lehner. 1981. Immunology of Oral Diseases, pp. 464, Blackwell Scientific Publications, Oxford, UK.
35. Rossomando, E. F., E. Kousvelari, B. W. Janicki, L. A. Tabak. 2001. Improvement of oral health in the postgenomic era: opportunities for government/industry partnerships, Compend Contin Educ Dent 22:570-2, 574.
36. Schade, S., M. Jolley: U.S. Pat. No. 5,804,395 issued Sep. 8, 1998.
37. Schernhammer, E. S., F. Laden, F. E. Speizer, W. C. Willet, D. J. Hunter, I. Kawachi, C. S. Fuchs and G. A. Colditz. 2003. Night-Shift Work and Risk for Colorectal Cancer in the Nurse's Health Study. J Natl Cancer Inst, 95:825-828.
38. Seymour, E. 1995. Salivary sampling device and sample adequacy system: U.S. Pat. No. 5,393,496. Patent and Trademark Office, United States, Salivary Diagnostic Systems, Vancouver, Wash.
39. Sherman, K. E., R. L. Creager, J. O'Brien, S. Sargent, S. Piacentini, T. Thieme. 1994. The use of oral fluid for hepatitis C antibody screening, Am J Gastroenterol 89:2025-7.
40. Slots, J., M. A. Taubman. 1991. Contemporary oral microbiology and immunology, Mosby, St. Louis, Mo.
41. Soderling, E. 1989. Practical Aspects of Salivary Analysis, Human Saliva: Clinical Chemistry and Microbiology, 1-24. Vol. 1, Boca Raton, CRC Press.
42. Stevens, R. G. and M. S. Rea. 2001. Light in the Built Environment: Potential Role of Circadian Disruption in Endocrine Disruption and Breast Cancer. Cancer Causes Control, 12:279-287.
43. Streckfus C. F., L. R. Bigler. 2002. Saliva as a diagnostic fluid, Oral Dis 8:69-76.
44. Tabak L. A. 2001. A revolution in biomedical assessment: the development of salivary diagnostics, J Dent Educ 65:1335-9.
45. Tencza, S., K. Islam, V. Kalia, M. Nasir, M. Jolley, R. Montelaro. 2000. Development of a fluorescence polarization-based diagnostic assay for equine infectious anemia virus, J Clin Microbiol 38:1854-9.
46. Tenovuo J. 1989. Human Saliva: Clinical Chemistry and Microbiology, 256. Vol. 1, Boca Raton, Fla., CRC Press.
47. Terpetschnig E., H. Szmacinski, J. Lakowicz. 1995. Fluorescence polarization immunoassay of a high-molecular-weight antigen based on a long-lifetime Ru-ligand complex, Anal Biochem, 227:140-7.
48. Thieme T R, Goldstein A S, Piacentini S C, Klimkow N M: Oral Collection Device and Kit: U.S. Pat. No. 5,830,410. Patent and Trademark Office, United States, Epitope, Inc., Beaverton, Oreg., 1998.
49. Thieme T, Piacentini S, Davidson S, Steingart K. 1994. Determination of measles, mumps, and rubella immunization status using oral fluid samples, JAMA 272:219-21.
50. Tsigo, C. and G. P. Chrousos, 1996. Differential diagnosis and management of Cushing's syndrome, Annu. Rev. Med. 47: 443-461.
51. Watts, N. B., and J. H. Keffer. 1989. Adrenal Cortex in Practical Endocrinology, 4^{th} ed, pp 91-120, Lea and Febiger, Philadelphia, Pa.

Having described the invention, one of skill in the art will appreciate in the appended claims that many modifications and variations of the present invention are possible in light of the above teachings. It is therefore, to be understood that, within the scope of the appended claims, the invention may be practiced otherwise than as specifically described.

What is claimed is:

1. A competitive method for estimating the concentration of a stress biomarker in a sample, comprising the steps:
a. intermixing said sample suspected of containing said biomarker with a specific antibody to said biomarker protein and a competitive biomarker reagent labeled with a fluorochrome capable of binding to said specific antibody to produce a mixture;
b. incubating said mixture for 15 seconds to 5 minutes;
c. detecting the binding interaction of said biomarker and antibody;
d. quantitating the concentration of the biomarker from said detected binding interaction of said biomarker and said antibody.

2. The method of claim 1, wherein said detection of said binding interaction is by a change in fluorescence polarization.

3. The method of claim 1, wherein said detection of said binding interaction is by a change in fluorescence lifetime.

4. The method of claim 1, wherein said detection of said binding interaction is by sensitized fluorescence of the acceptor or by quenching of donor fluorescence or by fluorescence depolarization.

5. The method of claim 1, wherein said method comprises the additional steps of:
e. measuring the fluorescence polarization of a negative control solution known not to contain said biomarker, a positive control solution with a known concentration of said biomarker or both, and;
f. comparing the measured concentration of said mixture with the measured fluorescence polarization of said negative control solution, said positive control solution, or both.

6. The method of claim 1, wherein said stress biomarker is selected from the group consisting of cortisol, melatonin and secretory IgA.

7. The method of claim 1 wherein said fluorochrome is pH independent.

8. The method of claim 1 wherein said fluorochrome is selected from the group consisting of 7-AAD, Acridine Orange, Alexa 488, Alexa 532, Alexa 546, Alexa 568, Alexa 594, Aminonapthalene, Benzoxadiazole, BODIPY 493/504, BODIPY 505/515, BODIPY 576/589, BODIPY FL, BODIPY TMR, BODIPY TR, Carboxytetramethylrhodamine, Cascade Blue, a Coumarin, Cy2, CY3, CY5, CY9, Dansyl Chloride, DAPI, Eosin, Erythrosin, Ethidium Homodimer II, Ethidium Bromide, Fluorescamine, Fluorescein, FTC, GFP (yellow shifted mutants T203Y, T203F, S65G/S72A), Hoechst 33242, Hoechst 33258, IAEDANS, an Indopyras Dye, a Lanthanide Chelate, a Lanthanide Cryptate, Lissamine Rhodamine, Lucifer Yellow, Maleimide, MANT, MQAE, NBD, Oregon Green 488, Oregon Green 514, Oregon Green 500, Phycoerythrin, a Porphyrin, Propidium Iodide, Pyrene, Pyrene Butyrate, Pyrene Maleimide, Pyridyloxazole, Rhodamine 123, Rhodamine 6G, Rhodamine Green, SPQ, Texas Red, TMRM, TOTO-1, TRITC, YOYO-1, vitamin B12, flavin-adenine dinucleotide, and nicotinamide-adenine dinucleotide.

9. The method of claim 1 wherein said fluorochrome concentration is 1 nM or less and the sample millipolarization is increased or decreased by at least 10 mp.

10. The method of claim 1 wherein the said antibody is polyclonal or monoclonal.

11. The method of claim 1, wherein said sample is obtained from bodily fluids selected from the group consisting of saliva, oral rinse expectorant, oral fluid including oral mucosal transudate and gingival crevicular fluid, urine, sweat, tears, blood, serum, stool, gastric fluid, synovial fluid, phlegm, and other clinical and laboratory specimens and samples.
